(19) United States
(12) Patent Application Publication
(10) Pub. No.: US 2005/0272772 A1
(43) Pub. Date: Dec. 8, 2005

(54) Pharmaceutical compositions for the treatment of pruritus

(76) Inventor: Jonathan J. Burbaum, San Diego, CA (US)

Correspondence Address: PRIEST & GOLDSTEIN PLLC, 5015 SOUTHPARK DRIVE, SUITE 230, DURHAM, NC, 27713-7736, US

(21) Appl. No.: 11/145,134

(22) Filed: Jun. 3, 2005

Related U.S. Application Data
(60) Provisional application No. 60/576,527, filed on Jun. 3, 2004.

(51) Int. Cl. A61K 31/445; A61K 31/24; A61K 31/16

(52) U.S. Cl. 514/317; 514/537; 514/625

(57) ABSTRACT

Composition comprising: a carrier; a first active ingredient comprising capsaicin, a capsaicin analog, another vanilloid receptor agonist or mixtures thereof; and at least one second active ingredient that functions as a topical anesthetic to cause localized numbness to induced skin irritation. Technique for treating the symptoms of pruritus comprising the step of applying a composition comprising a carrier, a first active ingredient comprising capsaicin, a capsaicin analog, another vanilloid receptor agonist or mixtures thereof, and at least one second active ingredient that functions as a topical anesthetic, to cause localized numbness to induced skin irritations.

### RELATED APPLICATION

The present invention claims the benefit of U.S. Provisional Application Ser. No. 60/576,527 filed Jun. 3, 2004, which is incorporated by reference herein in its entirety.

### FIELD OF THE INVENTION

The present invention relates to pharmaceutical compositions for the symptomatic treatment of pruritus (itch).

### BACKGROUND OF THE INVENTION

Chronic pruritus (itch) is a common symptom that relates to many different disease states, including pruritus ani, scabies, mite infestation, pediculosis, insect bites, eczema (both contact and atopic), urticaria (hives), dermographism, prickly heat, lichen planus, dermatitis herpetiformis, and toxic eruption. The neurological basis of pruritus is poorly understood, but is thought to be carried by a set of histamine-sensitive nerve fibers distinct from capsaicin-sensitive nerve fibers that carry pain signals (Schmelz et al. 1997, J Neurosci 17: 8003-8008; Schmelz et al. 2003, J Neurophysiol 89: 2441-2448).

Despite the classification of histamine-sensitive neurons as those that confer the sensation of itch, topical antihistamines are not effective in many cases of pruritus. In fact, topical antihistamines may sensitize the skin and result in allergic contact dermatitis. Further, despite the classification of capsaicin-sensitive neurons as those that confer pain, capsaicin has been shown to be effective in a number of cases of persistent itching, especially pruritus ani.

Pruritus ani is an extremely common proctologic problem, characterized by intense itching localized to the anus and perianal skin. Pruritus ani may result from an underlying disorder of the epithelium in the anal and perianal area or from anorectal pathology, or a host of other conditions [Taylor R B, ed. Family Medicine: Principles & Practice. 5th ed. New York: Springer-Verlag, 1998:792]. However, in many patients with pruritus ani there is no discernible cause for the condition. Fecal contamination of the perineum in the absence of gross soiling, irritant chemicals in the feces, and allergies to locally applied agents or components of diet have been implicated, but not conclusively proved to be of relevance [Jones D. J., BMJ (1992) 305:575-7]. Psychosomatic etiology has also been suggested [Jones (1992) ibid]. When no demonstrable cause is found, the phenomenon is often described as idiopathic and general advice regarding hygiene and drying methods is given, which results in relief only for some patients [Mazier, W. P., Surg Clin North Am (1994) 74: 1277-92].

Capsaicin is a natural product derived from plants of the Solanaceae family. Topical application of capsaicin is known to be safe and effective in the treatment of pain and itching [Hautkappe, M., et al., Clin J Pain (1998) 14(2):97-106]. Although the precise mechanism of action is not fully understood, evidence suggests that capsaicin is a neuropeptide-active agent that affects synthesis, storage, transport, and release of substance P [Burks, T. F., et al., Fed Proc (1985) 44(9):2531-4]. Substance P is thought to be an important chemical mediator of pain and itching impulses from the periphery to the central nervous system [Greaves, M. W. and Wall, P. D., Lancet (1996) 5348(9032):938-40; Rumsfield, J. A. and West, D. P., DICP (1991) 25(4):381-7].

Unfortunately, although capsaicin is often the most effective agent available, it is also a potent skin irritant and produces an uncomfortable burning sensation to the skin. Although prescribed frequently, capsaicin is used to only a limited extent due to this unpleasant side effect.

A capsaicin or a capsaicin analog based composition also containing a pain reliever which does not intolerably irritate the skin or cause a burning discomfort would be desirable to patients who are experiencing the discomfort of pruritus.

### SUMMARY OF THE INVENTION

In one embodiment, the present invention provides a composition comprising: a carrier; a first active ingredient comprising capsaicin, a capsaicin analog, another vanilloid receptor agonist or mixtures thereof; and at least one second active ingredient that functions as a topical anesthetic to cause localized numbness to alleviate capsaicin induced skin irritation.

In another embodiment, the present invention provides a method for treating the symptoms of pruritus comprising the step of applying a composition comprising a carrier, a first active ingredient comprising capsaicin, a capsaicin analog, another vanilloid receptor agonist or mixtures thereof, and at least one second active ingredient that functions as a topical anesthetic, to cause localized numbness to capsaicin induced skin irritations.

In a further embodiment, the present invention provides a method for making a composition useful for topical application to treat pruritus, said method comprising mixing a carrier with a first active ingredient comprising capsaicin, a capsaicin analog, another vanilloid receptor agonist or mixtures thereof, and at least one second active ingredient to induce numbness to capsaicin induced skin irritation to form a solution containing in the range of 0.001 to 0.25 percent by weight of the first active ingredient, wherein the second active ingredient comprises a topical anesthetic.

A more complete understanding of the present invention, as well as other embodiments, features and advantages of the invention will be apparent from the following detailed description.

### DETAILED DESCRIPTION OF THE INVENTION

The present invention relates to pharmaceutical compositions for the topical treatment of pruritus, especially idiopathic pruritus ani, comprising as active ingredients capsaicin or a capsaicin analog and a topical anesthetic.

Pruritus ani as an exemplary form of pruritus is a common and embarrassing proctologic problem. The symptoms of idiopathic pruritus ani can be intractable and the condition is difficult to treat. Itching of the anus is a common condition afflicting up to 5% of the population [Mazier, (1994) ibid.; Hanno, R. & Murphy, P. (1987) Dermatologic Clinics 5, 811-816]. The incidence of primary and secondary pruritus ani has varied between different studies. Several studies showed that the incidence of idiopathic pruritus ani is 75 to 95 percent of reported cases [Bernardi, R. S. and Chen, H. P., Surg Gynecol Obstet (1988) 167:359]. Other authors found that the incidence is only 25 percent [Wexner, S. D. and Daily, T. H., Curr Concepts Skin Disorders (1986) 7:5-9]. Specific etiologic factors can be addressed on an individual basis, usually resulting in temporary symptomatic relief. The majority of patients with idiopathic pruritus ani respond favorably to conservative treatment, such as dietary and hygienic advice and steroid-containing medication. Previous attempts to treat intractable pruritus ani include perianal injection of anesthetic agents [Gabriel, W. B., BMJ (1929) I: 1070-2], surgical disruption of the sensory nerve supply to the perineal area [Bacon, H. E., Anus Rectum and Sigmoid Colon: Diagnosis and Treatment. 3^{rd} Ed. Vol. 1. Philadelphia, Pa.: J. B. Lippincott, 1949], cryotherapy [Detrano, S. J., J Dermatol Surg Oncol (1984) 10:483-4], methylene blue injection [Farouk, R. and Lee, P. W. R., BMJ (1997) 84:670], and even hypnotherapy [Rucklidge, J. J. and Saunders, D., Psychosm Med (1999) 61(3):355-358]. Most of these attempts have had limited success and significant side effects, especially for chronic use.

Capsaicin is known to be effective in the treatment of pain, and this property is attributed to its ability to deplete substance P from capsaicin-sensitive afferent peripheral neurons. It has also been indicated that various skin itching phenomena may be alleviated by treatment with capsaicin, although itch signaling is generally believed to be mediated by histamine-sensitive afferent peripheral neurons. Capsaicin is a naturally occurring compound extracted, for example, from red chili peppers. Its pharmacological action for pain relief is depletion of substance P from sensory neurons, but its action against pruritus is not well understood. Capsaicin binds specifically to type-C sensory neurons, inducing release of substance P followed by inhibition of synthesis, transport and storage of substance P [Greaves and Wall, ibid.]. Topical application of capsaicin is known to be safe and effective for relief of pain associated with postherpetic neuralgia, rheumatoid arthritis and several other painful conditions. It also has been shown that capsaicin is effective in the treatment of histamine-induced pruritus, aquagenic pruritus, itching associated with uremia, nodular prurigo and postmastectomy syndrome [Hautkappe, M., et al., ibid.]. In addition to capsaicin, analog compounds exhibiting the same types of activity include, for example, civamide (cis-8-methyl-N-vanillyl-5 nonenamide), dihydrocapsaicin (8-methyl-N-vanillyl-nonamide), nordihydrocapsaicin (7-methyl-N-vanillyl-octamide), homodihydrocapsaicin (9-methyl-N-vanillyl-decamide), and homocapsaicin (trans-9-methyl-N-vanillyl-7-decenamide). These analogs and others having such activity and having chemical structures closely related to capsaicin can be substituted for capsaicin according to the teachings herein. In addition, there are other classes of similarly useful vanilloid receptor agonists that possess similar pharmacological activity, which include, for example, resiniferatoxin and tinyatoxin. The term “capsaicin” as used with respect to the compositions and methods herein includes such analogs.

Topical application of capsaicin cream ranging from concentrations of 0.006 to 1% percent was previously used to treat itching. No serious side effects were noted, except a painful, burning sensation at the site of application, especially during the first days of use [Lotti, T., et al., J Am Acad Dermatol (1994) 30(2 pt 1):232-5]. However, these side effects could still prevent up to about 30 percent of potential patients from continuing capsaicin treatment [Yosipovitch, G. and David, M., Medicine Update (1998) 18:11-17].

Capsaicin is the compound, trans-8-methyl-N-vanillyl-5 nonenamide, a naturally occurring alkyl vanillylamide, a type of capsaicinoid. Capsaicin is found in high concentration in the fruit of plants of the Capsicum genus. The chili pepper, red pepper and paprika are all species of Capsicum. Capsicum is the dry powder obtained by grinding up the fruits of these plants. Capsicum oleoresin, also referred to as capsaicin oleoresin, is the liquid concentrate extracted from the dry powder. Capsaicin, a white crystalline compound, is obtained from the liquid concentrate. The capsaicin analogs as discussed above are either made as derivatives of capsaicin or otherwise conventionally synthesized.

The composition of the invention comprises capsaicin, a capsaicin analog, another vanilloid receptor agonist or mixtures thereof as a first active ingredient, and at least one topical anesthetic as a second active ingredient to numb the sensation of capsaicin induced skin irritation. The ingredients are contained in a carrier.

Generally speaking, the composition will contain a concentration within the range of between about 0.001% and about 0.25% by weight of capsaicin, capsaicin analog or other vanilloid receptor agonist. However, compositions containing less than about 0.001% by weight of capsaicin will provide a diminishing, but still therapeutic, effect. Even trace concentrations of capsaicin will provide a minute therapeutic effect. Compositions containing more than about 0.25% by weight of capsaicin, capsaicin analog or other vanilloid receptor agonist will also provide a therapeutic effect, except that the burning side effect will increase in proportion to the increased concentration of capsaicin. Compositions containing about 5% or more by weight of capsaicin, capsaicin analog or other vanilloid receptor agonist could be used, for example, but the burning sensation is considered intense. For these reasons, compositions containing a concentration within the range of between about 0.001% and about 0.25% by weight of capsaicin, capsaicin analog or other vanilloid receptor agonist are preferred. In another embodiment, a composition containing in the range of between about 0.001 and about 0.01%, most preferably about 0.006%, by weight of capsaicin, capsaicin analog or other vanilloid receptor agonist are employed. The selected concentration of capsaicin may further depend upon the patient's age, body weight, and the mode of administration.

The present invention increases the amount of capsaicin, capsaicin analog or other vanilloid receptor agonist that can be administered comfortably. Generally speaking, a sufficient amount of the at least one second active topical anesthetic ingredient is mixed with the carrier to numb the local area of the skin where the capsaicin, capsaicin analog or other vanilloid receptor agonist is applied.

The preparation of pharmaceutical compositions is well known in the art and has been described in many articles and textbooks. See for example, Remington's Pharmaceutical Sciences, Gennaro, A. R. ed., Mack Publishing Company, Easton, Pa., 1990, and especially pp. 1521-1712 therein. See also for example, U.S. Pat. Nos. 5,736,519, 5,733,877, 5,554,378, 5,439,688, 5,418,219, 5,354,900, 5,298,246, 5,164,372, 4,900,549, 4,755,383, 4,639,435, 4,457,917, and 4,064,236. The active capsaicin and topical anesthetic agents used in the compositions of the present invention are preferably mixed with an excipient, carrier, diluent, and optionally, a preservative or the like, to constitute a pharmacologically acceptable vehicle as known in the art. See for example the above U.S. patents. Examples of excipients include glucose, mannitol, inositol, sucrose, lactose, fructose, starch, corn starch, microcrystalline cellulose, hydroxypropylcellulose, hydroxypropylmethylcellulose, polyvinyl pyrrolidone and the like. Optionally, a thickener may be added, such as a natural gum, a cellulose derivative, an acrylic or vinyl polymer, or the like.

The pharmaceutical composition may be provided in liquid or semi-solid form. The liquid preparation is provided preferably as oil suspension, fat emulsion or microcapsule composition. A semi-solid composition is provided preferably as a hydrous or oily gel, or an ointment.

An oil suspension may be prepared by suspending or emulsifying capsaicin in an oleaginous base, such as sesame oil, olive oil, corn oil, soybean oil, cottonseed oil, peanut oil, lanolin, petroleum jelly, paraffin, Isopar, silicone oil, fatty acids of 6 to 30 carbon atoms or the corresponding glycerol or alcohol esters. Buffers for such suspensions include Sorensen buffer [Ergeb Physiol, 12393 (1912)], Clark-Lubs buffer [J Bact, 2(1):109, 191 (1917)], Macllvaine buffer [J Biol Chem, 49:183 (1921)], Michaelis buffer (Die Wasserstoffinonenkonzentration, p. 186 (1914)], and Kolthoff buffer [Biochem Z, 179:410 (1926)].

Fat emulsions may be prepared by adding to a fat or oil about 0.1-2.4% by weight of an emulsifier such as a phospholipid. An emulsifying aid and a stabilizer are also added, and the ingredients are mixed with heating, and solvents are removed. Water and an isotonic agent are added, and optionally the pH is adjusted. The mixture is then homogenized. Preferably, such fat emulsions contain an electric charge adjusting agent, such as acidic phospholipids, fatty acids, bilic acids, and salts thereof. Acidic phospholipids include phosphatidylserine, phosphatidylglycerol, phosphatidylinositol, and phosphatidic acid. Bilic acids include deoxycholic acid and taurocholic acid. The preparation of such pharmaceutical compositions is described in U.S. Pat. No. 5,733,877.

The composition may also be prepared as a hydrous gel. A hydrous gel base is dissolved or dispersed in aqueous solution containing a buffer and the active agents, and the solution is warmed or cooled to give a stable gel.

As indicated above, the active capsaicin may also be contained in oil droplets, and this form may be particularly useful for aerosol or spray preparations. For this purpose the capsaicin may be contained also in liposomes, microcapsules or nanoparticles.

Preferably, the carrier is water-based and forms an aqueous solution containing the other ingredients. An oil-based carrier solution containing the ingredients is an alternative to the aqueous carrier solution. Either aqueous or oil-based solutions further contain thickening agents to provide the composition with the viscosity of a liniment, cream, ointment, gel, or the like. Suitable thickening agents are well known.

Alternative embodiments of the present invention can also use a solid carrier containing the active ingredients. This enables the alternative embodiments to be applied via a stick applicator, patch or suppository. The solid carrier further contains thickening agents to provide the composition with the consistency of wax or paraffin.

The composition of the invention will often also contain a polyol, such as a polyol selected from the group consisting of propylene glycol, glycerine, polyethylene glycol, butylene glycol, and triethanolamine. Other ingredients such as inositol, methyl paraben, propyl paraben, Carbomer 940 and DL-panthenol may be included if desired.

The at least one second active topical anesthetic ingredient can be of various functionalities. In general, any topical anesthetic effectively absorbed by mucus membranes and compatible with capsaicin and capsaicin analogs, can be employed. The topical anesthetic is present in a concentration effective to induce localized numbness in the skin surface being treated by capsaicin. For example, the composition can comprise a topical anesthetic in an amount within a range of between about 1% and about 10% by weight, preferably between about 1% and about 5% by weight. The second active ingredient can be selected, for example, from the group consisting of pramoxine (Pramocaine®; Proxazocain®), articaine (Articaine®), prilocaine (Citanest®), etidocaine (Duranest®), 1-(4-butoxyphenyl)-3-(1-piperidinyl)-1-propanone (Dyclonine®), benzocaine (Hurricaine®; Lollicaine®; Cetacaine®; Gingicaine®; Comfortcaine®; and Topicale®), bupivacaine (Marcaine®), carbocaine (Mepivacaine®); propoxycaine (Ravocaine®), and tetracaine (Pontocaine®). In another embodiment, the second ingredient can be lidocaine (Lidoderm®). Of the above anesthetics, prilocaine, bupivacaine and tetracaine are preferred. Also preferred is EMLA (a 50-50 prilocaine/lidocaine mixture).

Further background information regarding capsaicin is provided in the following documents which are hereby incorporated herein by reference in their entirety: Lysy, J. et al., “Topical capsaicin—a novel and effective treatment for idiopathic intractable pruritus ani: a randomised, placebo controlled, crossover study”, Gut 2003, Volume 52, pages 1323-1326; and Anand, P., “Capsaicin and menthol in the treatment of itch and pain: recently cloned receptors provide the key”, Gut 2003, Volume 52, pages 1233-1235.

The invention also provides a method of treating pruritus in a patient in need of such treatment. A pharmaceutically effective amount of capsaicin, capsaicin analog or other vanilloid receptor agonist is topically administered to the patient together with a pharmaceutically effective amount of a topical anesthetic. The capsaicin, capsaicin analog or other vanilloid receptor agonist is preferably contained in a pharmaceutical composition of the invention. In the method of the invention, the symptoms of pruritus are treated by applying the above described composition topically to the skin. Generally speaking, the inventive composition, preferably in ointment or cream form, is applied to the area and rubbed in. The amount applied is not critical. Generally, the composition should be applied in an amount which is sufficient to wet the area of application. For example, in the treatment of pruritus ani, the amount used will typically be in the range of from about 0.3 to about 3 cubic centimeters.

The application of the composition can be repeated as required to control the discomfort. When the preferred composition of the invention is applied, nearly immediate relief is induced without a strong burning sensation. The relief lasts for up to 24 to 48 hours. The topical anesthetic improves patient compliance with the capsaicin treatment regimen. The knowledge that localized numbness will be induced encourages initial use, and the reduced pain then experienced encourages continued application.

### EXAMPLE

A composition made in accordance with one embodiment of the invention contains the following ingredients.

| Ingredient | wt. % |
|---|---|
| deionized water | 82.45 |
| propylene glycol | 5.00 |
| glycerine | 3.00 |
| polyethylene glycol | 1.00 |
| butylene glycol | 1.00 |
| triethanolamine | .60 |
| inositol | .20 |
| methyl paraben | .10 |
| propyl paraben | .10 |
| Carbomer 940 | .30 |
| DL-Panthenol | 1.00 |
| lidocaine | 5.00 |
| capsaicin or capsaicin analog | 0.25 |

While the present invention has been disclosed in a presently preferred context, it will be recognized that the present teachings may be adapted to a variety of contexts consistent with this disclosure and the claims that follow.

1. A composition comprising:
a carrier;
a first active ingredient comprising between about 0.001 and about 0.01% by weight of capsaicin, a capsaicin analog, another vanilloid receptor agonist or mixtures thereof; and
a second active ingredient comprising between about 1% and about 10% by weight of a topical anesthetic selected from the group consisting of pramoxine, articaine, prilocaine, etidocaine, 1-(4-butoxyphenyl)-3-(1-piperidinyl)-1-propanone, benzocaine, bupivacaine, carbocaine, propoxycaine, tetracaine, and EMLA.

2. The composition of claim 1 wherein the second active ingredient is prilocaine, bupivacaine or tetracaine.

3. The composition of claim 1 wherein the second active ingredient is EMLA.

4. The composition of claim 1 comprising between about 1% and about 5% by weight of a topical anesthetic.

5. The composition of claim 1 comprising an aqueous carrier.

6. The composition of claim 1 comprising an oil based carrier.

7. The composition of claim 1 comprising a solid suppository.

8. The composition of claim 1 further comprising a thickening agent to provide the composition with the consistency of a carrier selected from the group consisting of a liniment, cream, gel, and ointment.

9. The composition of claim 1 comprising at least one binding agent for binding the first active ingredient.

10. The composition of claim 1 comprising a polyol selected from the group consisting of propylene glycol, glycerine, polyethylene glycol, butylene glycol, and triethanolamine.

11. The composition of claim 1 wherein the first active ingredient is selected from the group consisting of civamide, dihydrocapsaicin, nordihydrocapsaicin, homodihydrocapsaicin, homocapsaicin, resiniferatoxin and tinyatoxin.

12. The composition of claim 7 comprising a two-part suppository, one part containing the anesthetic, the other containing capsaicin, a capsaicin analog, another vanilloid receptor agonist or mixtures thereof.

13. A method for treating the symptoms of pruritus comprising the step of applying a composition comprising a carrier, at least one first active ingredient comprising capsaicin, a capsaicin analog, another vanilloid receptor agonist or mixtures thereof, and at least one second active ingredient that functions as a topical anesthetic, to cause localized numbness to induced skin irritations.

14. The method of claim 13 in which the first active ingredient is at a concentration in the range of between about 0.001% and about 0.25% by weight.

15. The method of claim 13 in which a topical anesthetic at a concentration in the range between about 1% and about 10% by weight is employed.

16. The method of claim 13 in which the topical anesthetic is selected from the group consisting of pramoxine, articaine, prilocaine, etidocaine, 1-(4-butoxyphenyl)-3-(1-piperidinyl)-1-propanone, benzocaine, lidocaine, bupivacaine, carbocaine, propoxycaine, tetracaine, and EMLA.

17. The method of claim 13 in which an aqueous carrier is employed.

18. The method of claim 13 in which the composition further comprises a thickening agent to provide the composition with the consistency of a carrier selected from the group consisting of a liniment, cream, gel, and ointment.

19. The method of claim 13 in which the composition further comprises at least one binding agent for binding the first active ingredient.

20. The method of claim 13 in which the composition further comprises a polyol selected from the group consisting of propylene glycol, glycerine, polyethylene glycol, butylene glycol, and triethanolamine.

21. The method of claim 13 in which pruritus ani is treated.

22. The method of claim 14 in which the first active ingredient is at a concentration in the range of about 0.001% to about 0.01% by weight.

23. A method for making a composition useful for topical application to treat pruritus, said method comprising mixing a carrier with a first active ingredient comprising capsaicin, a capsaicin analog, another vanilloid receptor agonist or mixtures thereof, and at least one second active ingredient to induce numbness to induced skin irritation to form a solution containing in the range of from about 0.001 to about 0.25 percent by weight of the first active ingredient, wherein the second active ingredient comprises a topical anesthetic.

24. The method of claim 15 in which a topical anesthetic at a concentration in the range between about 1% and about 5% by weight is employed.

25. The method of claim 13 wherein the first active ingredient is selected from the group consisting of civamide, dihydrocapsaicin, nordihydrocapsaicin, homodihydrocapsaicin, homocapsaicin, resiniferatoxin and tinyatoxin.
